(19) United States
(12) Patent Application Publication
(10) Pub. No.: US 2025/0043329 A1
(43) Pub. Date: Feb. 6, 2025

(54) METHODS OF HIGH THROUGHPUT EPIGENOMIC MAPPING OF NORMAL AND MALIGNANT HEMATOPOIETIC STEM AND PROGENITOR CELLS

(71) Applicant: The Trustees of Columbia University in the City of New York, New York, NY (US)

(72) Inventor: Aaron D. VINY, New York, NY (US)

(21) Appl. No.: 18/791,198

(22) Filed: Jul. 31, 2024

Related U.S. Application Data
(60) Provisional application No. 63/516,797, filed on Jul. 31, 2023.

(51) Int. Cl. C12Q 1/6806

(52) U.S. Cl. CPC C12Q 1/6806

(57) ABSTRACT

Disclosed are methods of epigenetic mapping with low input capabilities that enable applications to human tissues. The method can also be incorporated with automated/semi-automated capabilities to enable the use epigenetic mapping in prospective clinical trials. The methods comprise shearing the isolated chromatin using focused ultrasonication technology, preferably at least twice, prior to immunoprecipitation of the sheared chromatin and isolation for subsequent DNA analysis.

### RELATED APPLICATIONS

This application claims the benefit of U.S. provisional patent application 63/516,797, filed Jul. 31, 2023 to Aaron D. Viny, titled “METHODS OF HIGH THROUGHPUT EPIGENOMIC MAPPING OF HEMATOPOIETIC STEM AND PROGENITOR CELLS,” the entirety of the disclosure of which is hereby incorporated by this reference.

### TECHNICAL FIELD

This document relates to reproducible, high throughput methods for determining chromatin structure and chromatin modifications in low cell input samples in small sample volumes by the use of focused bursts of ultrasonic acoustic energy for chromatin shearing before immunoprecipitation in a single pot format.

### BACKGROUND

Myelodysplastic syndromes (MDS) are a group of cancers in which immature blood cells in the bone marrow do not mature or become healthy blood cells. Acute myeloid leukemia (AML) is a type of cancer in which the bone marrow makes a large number of abnormal blood cells. AML is a complex disease with several etiologies characterized by incomplete cellular differentiation and clonal proliferation of hematopoietic stem and progenitor cells (HSPCs) in place of normal hematopoiesis. Mapping the epigenetic landscape of MDS and AML is essential for understanding gene regulatory mechanisms during cell fate changes, disease onset, progression, as well as for optimizing therapeutic regime.

Non-mutational epigenetic reprogramming is an emerging hallmark and enabling characteristic of cancer pathogenesis. Epigenetic regulation of gene activity is an essential component of chromatin stability and maintenance of proper cellular function and differentiation. Modifications of gene and histone landscape methylation and acetylation, three-dimensional chromatin structure, and protein-controlled activating and silencing switches are all regulated by epigenetic components, and thus are integral to understanding gene regulatory mechanisms during cell fate changes, disease onset, progression, as well as for optimizing therapeutic regime. In addition, this information holds immense potential for accurate identification of disease biomarkers, cell of origin, and targets for epigenetic therapy.

Unfortunately, existing chromatin immunoprecipitation sequencing (ChIP-Seq) workflows require significant cellular material and are very time-consuming, requiring cumbersome optimization. Moreover, cellular heterogeneity can influence accuracy of results from the perspective of sample preparation efficiency and also population-biased effects. Both normal and malignant bone marrow are heterogeneous in cell type and stage of differentiation that leads to imprecise results with regard to epigenetic determinates. Thus, improved methods for understanding the epigenetic regulatory processes, by which normal and malignant hematopoiesis are governed are needed.

### SUMMARY

The present disclosure relates to methods of epigenomic mapping, even for low cell input samples, using acoustic enhanced immunoprecipitation. Also disclosed herein are methods of isolating chromatin from small volume and small number of cells using acoustic enhanced immunoprecipitation. This approach enables a standardized, robust, automatable, half-day workflow for both ChIP and Hi-ChIP applications.

In one aspect, the present disclosure provides methods of assessing epigenetic regulation in a tissue. The methods comprise the steps of isolating primary cells from the tissue to produce a primary cell sample, wherein the primary cell sample comprises at least 5,000 primary cells, isolating chromatin from the primary cell sample, shearing the isolated chromatin using focused ultrasonication, immunoprecipitating the sheared chromatin and isolating the sheared chromatin for DNA analysis. The shearing using focused ultrasonication may include the application of the focused ultrasonication at least twice. In certain exemplary embodiments, the methods comprise 5,000-500,000 or 10,000-20,000 primary cells.

In a second aspect, the present disclosure provides methods for isolating chromatin. The methods comprise the steps of isolating primary cells from tissue to produce a primary cell sample, wherein the primary cell sample comprises at least 5,000 primary cells; isolating chromatin from the primary cell sample; shearing the isolated chromatin using focused ultrasonication technology twice; immunoprecipitating the sheared chromatin, and isolating the sheared chromatin for DNA analysis. In other exemplary aspects of the disclosure, the primary cell sample comprises 10,000-20,000 primary cells, at least 20,000 primary cells, or 5,000-50,000 primary cells. In an exemplary embodiment, the primary cell sample is isolated from bone marrow of a mammalian subject.

In a third aspect, the present disclosure provides methods for assessing epigenetic regulation in hematopoietic cells. The methods comprise providing a primary cell sample comprising at least 5,000 hematopoietic cells; isolating chromatin from the primary cell sample;

shearing the isolated chromatin using focused ultrasonication technology; immunoprecipitating the sheared chromatin; and isolating the sheared chromatin for DNA analysis. The shearing using focused ultrasonication may include the application of the focused ultrasonication at least twice. In certain embodiments, the primary cell sample comprises 10,000-20,000 primary cells, at least 20,000 primary cells, or 5,000-50,000 primary cells. In one exemplary embodiment, the primary cell sample is isolated from bone marrow of a mammalian subject.

In a fourth aspect, the present disclosure provides methods for isolating chromatin. The methods comprise the steps of isolating primary cells from bone marrow to produce a primary cell sample, wherein the primary cell sample comprises at least 5,000 bone marrow cells; isolating chromatin from the primary cell sample; shearing the isolated chromatin using focused ultrasonication technology at least twice; immunoprecipitating the sheared chromatin; and isolating the sheared chromatin for DNA analysis. The DNA analysis can be done with different methods, for example, PCR or next-generation sequencing. In one exemplary aspect, the primary cell sample comprises 5,000-50,000 primary cells. In some exemplary embodiments, the isolated chromatin is sheared using focused ultrasonication technology for a total of at least 30 minutes or, for a total of at least four hours.

The foregoing and other aspects, features, and advantages will be apparent from the DESCRIPTION and DRAWINGS, and from the CLAIMS if any are included.

### BRIEF DESCRIPTION OF THE DRAWINGS

Implementations will hereinafter be described in conjunction with the appended and/or included DRAWINGS.

FIGS. 1A and 1B depict, in accordance with certain embodiments, a schematic of the process for harvesting, extracting stem and progenitor cells from the bone marrow of a wild type mouse (FIG. 1A) and an exemplary schematic for high input and regular ChIP processing of hematopoietic cells (FIG. 1B).

FIGS. 2A-2D depict graphs of whole bone marrow cells, crosslinked, and sheared using Covaris® truChIP® and LE220Rsc (FIG. 2A), the same sample as in FIG. 2A post-CTCF IP (FIG. 2B), FACS-sorted stem/progenitor cells, crosslinked, and sheared using Covaris® truChIP® and LE220Rsc (FIG. 2C), and the same sample as in FIG. 2C, post-CTCF IP (FIG. 2D). FIG. 2A depicts results for 50,000 whole bone marrow cells which have been crosslinked and sheared using Covaris truChIP and LE220Rsc, and the chromatin yield is 21 ng. FIG. 2B depicts results for the sample as FIG. 2A though after post-CTCP IP, and the chromatin yield then is 8.42 ng. FIG. 2C depicts results for 50,000 stem/progenitor cells which have been crosslinked and sheared using Covaris truChIP and LE220Rsc, and the chromatin yield is 25.8 ng. FIG. 2D depicts results for the sample as FIG. 2C though after post-CTCP IP, and the chromatin yield then is 14.38 ng.

FIG. 3 is a representation of dynamic changes at the Mecom/Evil enhancer detected by the claimed methods. The Mecom/Evil enhancer loses ATAC accessibility and gains a differentially methylated region at a CTCF site within the Evil enhancer.

FIGS. 4A-4C depict, in accordance with certain embodiments, dynamic changes at the Mecom/Evil locus detected by the claimed methods.

### DETAILED DESCRIPTION

Detailed aspects and applications of the disclosure are described below in the following drawings and detailed description of the technology. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the disclosure. It will be understood, however, by those skilled in the relevant arts, that embodiments of the technology disclosed herein may be practiced without these specific details. It should be noted that there are many different and alternative configurations, devices and technologies to which the disclosed technologies may be applied. The full scope of the technology disclosed herein is not limited to the examples that are described below.

The singular forms “a,” “an,” and “the” include plural referents unless the context clearly dictates otherwise. Thus, for example, reference to “a step” includes reference to one or more of such steps.

The word “exemplary,” “example,” or various forms thereof are used herein to mean serving as an example, instance, or illustration. Any aspect or design described herein as “exemplary” or as an “example” is not necessarily to be construed as preferred or advantageous over other aspects or designs. Furthermore, examples are provided solely for purposes of clarity and understanding and are not meant to limit or restrict the disclosed subject matter or relevant portions of this disclosure in any manner. It is to be appreciated that a myriad of additional or alternate examples of varying scope could have been presented but have been omitted for purposes of brevity.

When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable. Throughout the description and claims of this specification, the words “comprise” and “contain” and variations of the words, for example “comprising” and “comprises”, mean “including but not limited to”, and are not intended to (and do not) exclude other components. As required, detailed embodiments of the present disclosure are included herein. It is to be understood that the disclosed embodiments are merely exemplary of the invention that may be embodied in various forms. Therefore, specific structural and functional details disclosed herein are not to be interpreted as limits, but merely as a basis for teaching one skilled in the art to employ the present invention. The specific examples below will enable the disclosure to be better understood. However, they are given merely by way of guidance and do not imply any limitation. The present disclosure may be understood more readily by reference to the following detailed description taken in connection with the accompanying figures and examples, which form a part of this disclosure. It is to be understood that this disclosure is not limited to the specific materials, devices, methods, applications, conditions, or parameters described and/or shown herein, and that the terminology used herein is for the purpose of describing particular embodiments by way of example only and is not intended to be limiting of the claimed inventions. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

As used herein, the term “DNA analysis” refers to techniques such as, but not limited to, quantitative PCR and next-generation sequencing.

The term “focused ultrasonication technology” refers to techniques that utilize high-frequency sound waves to create shear forces that can fragment nucleic acids, proteins and other material. Non-limiting examples are: Covaris® Adaptive Focused Acoustics® (AFA®), Diagenode Bioruptor®, and Qsonica Ultrasonics.

In certain aspects of the disclosure the methods are useful for assessing epigenetic regulation in a biological sample. A primary sample is produced by isolating primary cells from a tissue or by isolating cells from a cell line (such as a primary cell line). In certain embodiments, the primary cell sample comprises at least 5,000, 10,000-20,000, at least 20,000, or 5,000-50,000 primary cells. The primary cells from the tissue may be a normal cell or a malignant cells. In one aspect, the methods are used for assessing epigenetic regulation in hematopoietic cells. Thus, in certain embodiments, the primary cell comprises hematopoietic cells, for example, hematopoietic stem and/or progenitor cells. In one exemplary embodiment, the primary cell sample is isolated from bone marrow of a mammalian subject.

In other aspects disclosed herein, the methods are used to isolate chromatin. In one aspects, primary cells are isolated from a tissue producing a primary cell sample. In another aspects, the primary sample is produced by isolating cells from a cell line (such as a primary cell line). In some embodiments, the primary cell sample comprises 10,000-20,000, at least 20,000, or 5,000-50,000 primary cells. The primary cells may be a normal cell or a malignant cells. In some aspects, the primary cells are hematopoietic cell, for example, hematopoietic stem and/or progenitor cells. In some aspects, the methods are used for isolating chromatin from bone marrow sample.

Appropriate samples for use in the methods disclosed herein include any conventional biological sample obtained from an organism or a part thereof, such as a plant, animal, and the like.

In particular embodiments, the primary sample is a cell line, such as a primary cell line. The cell line can be treated or untreated. For example, the cell line can be treated with a drug candidate, compound, biologic agent, environmental stress, or genetic perturbation. In example embodiments, primary cells can be obtained from an animal model that expresses a CRISPR system (see, e.g., U.S. Pat. No. 11,197,467B2).

In particular embodiments, the biological sample is obtained from an animal subject, such as a human subject. A biological sample is any solid or fluid sample obtained from, excreted by or secreted by any living organism, including without limitation, single celled organisms, such as yeast, protozoans, and amoebas among others, multicellular organisms (such as plants or animals, including samples from a healthy or apparently healthy human subject or a human patient affected by a condition or disease to be diagnosed or investigated, such as cancer). For example, a biological sample can be a biological fluid obtained from, for example, blood, plasma, serum, urine, bile, ascites, saliva, cerebrospinal fluid, aqueous or vitreous humor, or any bodily secretion, a transudate, an exudate (for example, fluid obtained from an abscess or any other site of infection or inflammation), or fluid obtained from a joint (for example, a normal joint or a joint affected by disease, such as a rheumatoid arthritis, osteoarthritis, gout or septic arthritis). A sample can also be a sample obtained from any organ or tissue (including a biopsy or autopsy specimen, such as a tumor biopsy) or can include a cell (whether a primary cell or cultured cell) or medium conditioned by any cell, tissue, or organ. Exemplary samples include, without limitation, cells, cell lysates, blood smears, cyto-centrifuge preparations, cytology smears, bodily fluids (e.g., blood, plasma, serum, saliva, sputum, urine, bronchoalveolar lavage, semen, etc.), tissue biopsies (e.g., tumor biopsies), fine-needle aspirates, and/or tissue sections (e.g., cryostat tissue sections and/or paraffin-embedded tissue sections). In other examples, the sample includes circulating tumor cells (which can be identified by cell surface markers). In particular examples, samples are used directly (e.g., fresh or frozen), or can be manipulated prior to use, for example, by fixation (e.g., using formalin) and/or embedding in wax (such as formalin-fixed paraffin-embedded (FFPE) tissue samples). It will be appreciated that any method of obtaining tissue from a subject can be utilized, and that the selection of the method used will depend upon various factors such as the type of tissue, age of the subject, or procedures available to the practitioner. Standard techniques for acquisition of such samples are available. See, for example Schluger et al., J. Exp. Med. 176:1327-33 (1992); Bigby et al., Am. Rev. Respir. Dis. 133:515-18 (1986); Kovacs et al., NEJM 318:589-93 (1988); and Ognibene et al., Am. Rev. Respir. Dis. 129:929-32 (1984).

In example embodiments, the epigenetic states identified are correlated with a disease state or age-related state. In example embodiments, the epigenetic states identified are correlated with an environmental condition. The disclosed methods are also particularly suited to monitoring disease states, such as disease state in an organism, for example an animal subject, such as a mammalian subject, for example a human subject.

### Crosslinking

In some aspects, chromatin is fixed or crosslinked to preserve protein-DNA interactions as part of the step of isolating chromatin from the primary cell sample. In exemplary embodiments, proximity ligation or shearing is performed on crosslinked cells/nuclei to preserve spatial proximity relationships in the cell/nuclei. In some embodiments of the disclosed method, the nucleic acids present in the cell or cells are fixed in position relative to each other by chemical crosslinking, for example by contacting the cells with one or more chemical cross linkers. This treatment locks in the spatial relationships between portions of nucleic acids in a cell. Any method of fixing the nucleic acids in their positions can be used. In some embodiments, the cells are fixed, for example with a fixative, such as an aldehyde, for example formaldehyde or gluteraldehyde. In some embodiments, a sample of one or more cells is cross-linked with a cross-linker to maintain the spatial relationships in the cell. For example, a sample of cells can be treated with a cross-linker to lock in the spatial information or relationship about the molecules in the cells, such as the DNA and RNA in the cell. In other embodiments, the relative positions of the nucleic acid can be maintained without using crosslinking agents. For example, the nucleic acids can be stabilized using spermine and spermidine (see Cullen et al., Science 261, 203 (1993), which is specifically incorporated herein by reference in its entirety). Other methods of maintaining the positional relationships of nucleic acids are known in the art. In some embodiments, nuclei are stabilized by embedding in a polymer such as agarose. In some embodiments, the cross-linker is a reversible cross-linker. In some embodiments, the cross-linker is reversed, for example after the fragments are joined and the spatial information is locked in. In specific examples, the nucleic acids are released from the cross-linked three-dimensional matrix by treatment with an agent, such as a proteinase, that degrade the proteinaceous material from the sample, thereby releasing the nucleic acids for further analysis, such as determination of the nucleic acid sequence. In specific embodiments, the sample is contacted with a proteinase, such as Proteinase K. In some embodiments of the disclosed methods, the cells are contacted with a crosslinking agent to provide the cross-linked cells. In some examples, the cells are contacted with a protein-nucleic acid crosslinking agent, a nucleic acid-nucleic acid crosslinking agent, a protein-protein crosslinking agent or any combination thereof. By this method, the nucleic acids present in the sample become resistant to special rearrangement and the spatial information about the relative locations of nucleic acids in the cell is maintained. In certain embodiments, the cells are cross linked such that the cohesin complex is not denatured. In some examples, a cross-linker is reversible, such that the cross-linked molecules can be easily separated in subsequent steps of the method. In some examples, a cross-linker is a non-reversible cross-linker, such that the cross-linked molecules cannot be easily separated. In some examples, a cross-linker is light, such as UV light. In some examples, a cross linker is light activated. These cross-linkers include formaldehyde, disuccinimidyl glutarate, UV light, psoralens and their derivatives such as aminomethyltrioxsalen, glutaraldehyde, ethylene glycol bis [succinimidylsuccinate], bissulfosuccinimidyl suberate, 1-Ethyl-3-[3-dimethylaminopropyl] carbodiimide (EDC) bis [sulfosuccinimidyl] suberate (BS³) and other compounds known to those skilled in the art, including those described in the Thermo Scientific Pierce Crosslinking Technical Handbook, Thermo Scientific (2009) as available on the world wide web at piercenet.com/files/1601673_Crosslink_HB_Intl.pdf.

As used herein the term “contacting” refers to placement in direct physical association, including both in solid or liquid form, for example contacting a sample with a crosslinking agent or a probe. As used herein the term “Crosslinking agent” refers to a chemical agent or even light, which facilitates the attachment of one molecule to another molecule. Crosslinking agents can be protein-nucleic acid crosslinking agents, nucleic acid-nucleic acid crosslinking agents, and protein-protein crosslinking agents. Examples of such agents are known in the art. In some embodiments, a crosslinking agent is a reversible crosslinking agent. In some embodiments, a crosslinking agent is a non-reversible crosslinking agent.

### Isolated Nuclei

In some embodiments, the cells are lysed to release the cellular contents, for example after crosslinking, as part of the step of isolating chromatin from the primary cell sample. In some examples the nuclei are lysed as well, while in other examples, the nuclei are maintained intact, which can then be isolated and optionally lysed, for example using a reagent that selectively targets the nuclei or other separation technique known in the art.

In some examples, the primary cell sample is a sample of permeabilized nuclei, multiple nuclei, or isolated nuclei. In certain embodiments the cells are synchronized cells, (such at various points in the cell cycle, for example metaphase) before nuclei are isolated. In certain embodiments, cells are lysed under conditions that are non-denaturing, such that proteins remain folded in their native conformation and chromatin structure is maintained (see, e.g., US patent application publication US20240150830A1).

As used herein the term “isolated” refers to an “isolated” biological component (for example, an end-joined fragmented nucleic acids or nuclei as described herein) has been substantially separated or purified away from other biological components in the cell of the organism, in which the component naturally occurs, for example, extra-chromatin DNA and RNA, proteins and organelles. Nucleic acids and proteins that have been “isolated” include nucleic acids and proteins purified by standard purification methods, for example from a sample. The term also embraces nucleic acids and proteins prepared by recombinant expression in a host cell as well as chemically synthesized nucleic acids. It is understood that the term “isolated” does not imply that the biological component is free of trace contamination and can include nucleic acid molecules that are at least 50% isolated, such as at least 75%, 80%, 90%, 95%, 98%, 99%, or even 100% isolated.

### Permeabilizing Nuclei

In certain examples, the methods disclosed herein further comprises permeabilizing nuclei, for example, as part of the step of isolating chromatin from the primary cell sample. In certain embodiments, nuclei of the present invention can be permeabilized according to any method known in the art. In some cases, the nuclei may be permeabilized to allow access for nucleic acid processing reagents. The permeabilization may be performed in a way to minimally perturb the spatial proximity of nucleic acids, protein folding, organelles, and/or nuclei. In certain embodiments, the nuclei are permeabilized, such that protein complexes do not fall apart, or proteins are not denatured. In some instances, the cells may be permeabilized using a permeabilization agent. Examples of permeabilization agents include NP40, digitonin, tween, streptolysin, exonuclease 1 buffer (NEB) and pepsin, and cationic lipids. In other instances, the cells, organelles, and/or nuclei may be permeabilized using hypotonic shock and/or ultrasonication. In other cases, the nucleic acid processing reagents e.g., enzymes such as nuclease, polymerase and/or ligase, may be highly charged, which may allow them to permeabilize through the membranes of the nuclei. Other embodiments include use of cell penetrating peptides to deliver cargo to the nuclei and allow capture of material. In certain embodiments, permeabilization steps, including pre-permeabilization are automated.

In certain embodiments, nuclei are permeabilized with a detergent. In certain embodiments, the detergent is non-ionic. In certain embodiments, the concentration of the detergent is sufficient to permeabilize the nuclei without denaturing proteins in the nuclei. In certain embodiments, NP40, digitonin, or tween is used. For example, the concentration of detergent used herein may be from 0.005% to 1%, from 0.01% to 0.8%, from 0.01% to 0.6%, from 0.01% to 0.4%, from 0.01% to 0.2%, from 0.01% to 0.1%, from 0.005% to 0.05%, from 0.01% to 0.03%, from 0.015% to 0.025%, from 0.018% to 0.022%, from 0.015% to 0.017%, from 0.016% to 0.018%, from 0.017% to 0.019%, from 0.018% to 0.02%, from 0.019% to 0.021%, from 0.02% to 0.022%, or from 0.021% to 0.023%. In some cases, the concentration of the detergent may be about 0.01%, about 0.015%, about 0.02%, about 0.025%, or about 0.03%. For example, the concentration of the detergent may be about 0.02%. In certain embodiments, SDS is used at concentrations below 0.5%, such as 0.1, 0.05, or less than 0.01%. In certain embodiments, the nuclei are not heated during permeabilization.

### Fragmenting, End-Repair, Fill-In and Ligation

In some embodiments, in order to create discrete portions of nucleic acid that can be joined together in subsequent steps of the methods, the nucleic acids present in the cells, such as cross-linked cells, are fragmented. In some embodiments, chromatin is fragmented, such that chromatin bound by proteins are protected from cleavage. The fragmentation can be done by a variety of methods, such as enzymatic and chemical cleavage. For example, DNA can be fragmented using any DNA cutter or combination thereof, such as, MseI and Csp6I; MboI, MseI, NlaIII and Csp6I; DNase I; micrococcal nuclease (MNase); benzonase; cyanase; another restriction enzyme; or a transposase complex. In one example embodiment, chromatin can be fragmented with a transposase to insert adapters into fragmented chromatin, such as in ATAC-seq (see, e.g., Buenrostro, et al., Transposition of native chromatin for fast and sensitive epigenomic profiling of open chromatin, DNA-binding proteins and nucleosome position. Nature methods 2013; 10 (12): 1213-1218). In one example embodiment, DNA can be fragmented using an endonuclease that cuts a specific sequence of DNA and leaves behind a DNA fragment with a 5′ overhang, thereby yielding fragmented DNA. In other examples, an endonuclease can be selected that cuts the DNA at random spots and yields overhangs or blunt ends. In some embodiments, fragmenting the nucleic acid present in the one or more cells comprises enzymatic digestion with an endonuclease that leaves 5′ overhanging ends. Enzymes that fragment, or cut, nucleic acids and yield an overhanging sequence are known in the art and can be obtained from such commercial sources as New England BioLabs® and Promega®. One of ordinary skill in the art can choose the restriction enzyme without undue experimentation. One of ordinary skill in the art will appreciate that using different fragmentation techniques, such as different enzymes with different sequence requirements, will yield different fragmentation patterns and therefore different nucleic acid ends. The process of fragmenting the sample can yield ends that are capable of being joined.

In certain embodiments, the ends of the fragmented DNA is repaired (e.g., end repair). Commercial reagents and protocols are available for DNA end repair. Fragmentation of polynucleotide molecules may result in fragments with a heterogeneous mix of blunt and 3′- and 5′-overhanging ends. It is therefore desirable to repair the fragment ends using methods or kits known in the art to generate ends that are optimal for ligation, for example, blunt sites of chromatin fragments. In a particular embodiment, the fragment ends of the nucleic acids are blunt ended. One method of the invention involves repairing the fragment ends with nucleotide triphosphates and a nucleic acid polymerase. The nucleotide triphosphates may contain a labeling modification, for example biotin or similar protein binding ligand, that allows selection of the end repaired fragments. The polymerase may be Klenow DNA polymerase or similar nucleic acid polymerase, that may have exonuclease activity in order to remove any 3′ overhanging ends. The reaction may be carried out with all four nucleotides, of which 0-4 may carry labeling modifications. The reaction may be carried out with a single labelled nucleoside triphosphate, and three unlabeled triphosphates, or may be carried out with two, three or four labeled nucleotides.

As used herein the term “nucleic acid (molecule or sequence)” refers to a deoxyribonucleotide or ribonucleotide polymer including without limitation, cDNA, mRNA, genomic DNA, and synthetic (such as chemically synthesized) DNA or RNA or hybrids thereof. The nucleic acid can be double-stranded (ds) or single-stranded (ss). Where single-stranded, the nucleic acid can be the sense strand or the antisense strand. Nucleic acids can include natural nucleotides (such as A, T/U, C, and G), and can also include analogs of natural nucleotides, such as labeled nucleotides. Some examples of nucleic acids include the probes disclosed herein.

The major nucleotides of DNA are deoxyadenosine 5′-triphosphate (dATP or A), deoxyguanosine 5′-triphosphate (dGTP or G), deoxycytidine 5′-triphosphate (dCTP or C) and deoxythymidine 5′-triphosphate (dTTP or T). The major nucleotides of RNA are adenosine 5′-triphosphate (ATP or A), guanosine 5′-triphosphate (GTP or G), cytidine 5′-triphosphate (CTP or C) and uridine 5′-triphosphate (UTP or U). Nucleotides include those nucleotides containing modified bases, modified sugar moieties, and modified phosphate backbones, for example as described in U.S. Pat. No. 5,866,336 to Nazarenko et al.

Examples of modified base moieties which can be used to modify nucleotides at any position on its structure include, but are not limited to: 5-fluorouracil, 5-bromouracil, 5-chlorouracil, 5-iodouracil, hypoxanthine, xanthine, acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methyl cytosine, N6-adenine, 7-methylguanine, 5-methylaminomethyluracil, methoxyarninomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, uracil-S-oxyacetic acid, 5-methyl-2-thiouracil, 3-(3-amino-3-N-2-carboxypropyl) uracil, 2,6-diaminopurine and biotinylated analogs, amongst others.

Examples of modified sugar moieties which may be used to modify nucleotides at any position on its structure include, but are not limited to arabinose, 2-fluoroarabinose, xylose, and hexose, or a modified component of the phosphate backbone, such as phosphorothioate, a phosphorodithioate, a phosphoramidothioate, a phosphoramidate, a phosphordiamidate, a methylphosphonate, an alkyl phosphotriester, or a formacetal or analog thereof.

Ligation may be carried out in situ using any ligase known in the art and described further in the examples to obtain covalently linked joined DNA molecules. The ligation reaction may be carried out using any suitable ligase, for example, T3 or T4 ligase. Covalently linked: Refers to a covalent linkage between atoms by the formation of a covalent bond characterized by the sharing of pairs of electrons between atoms. In one example, a covalent link is a bond between an oxygen and a phosphorous, such as phosphodiester bonds in the backbone of a nucleic acid strand. In another example, a covalent link is one between a nucleic acid protein, another protein and/or nucleic acid that has been crosslinked by chemical means. In another example, a covalent link is one between fragmented nucleic acids.

In some embodiments, the end joined DNA that includes a labeled nucleotide is captured with a specific binding agent that specifically binds a capture moiety, such as biotin, on the labeled nucleotide. In some embodiments, the capture moiety is adsorbed or otherwise captured on a surface. In specific embodiments, the end target joined DNA is labeled with biotin, for instance by incorporation of biotin-14-CTP or other biotinylated nucleotide during the filling in of the 5′ overhang, for example with a DNA polymerase, allowing capture by streptavidin. This step can also be referred to herein as “biotin filling” or “biotin-fill-in”. In some embodiments, the step(s) of biotin filling can be completed in about 1 to about 45 minutes such as about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or about 45 minutes. Any additional biotin filing steps as discussed elsewhere herein, can also be completed in about in about 1 to about 45 minutes such as about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or about 45 minutes.

As used herein the term “biotin-14-CTP” refers to a biologically active analog of cytosine-5′-triphosphate that is readily incorporated into a nucleic acid by polymerase or a reverse transcriptase. In some examples, biotin-14-CTP is incorporated into a nucleic acid fragment that has a 3′ overhang.

As used herein the term “capture moieties” refers to molecules or other substances that when attached to a nucleic acid molecule, such as an end joined nucleic acid, allow for the capture of the nucleic acid molecule through interactions of the capture moiety and something that the capture moiety binds to, such as a particular surface and/or molecule, such as a specific binding molecule that is capable of specifically binding to the capture moiety.

Other means for labeling, capturing, and detecting nucleic acid probes include but are not limited to: incorporation of aminoallyl-labeled nucleotides, incorporation of sulfhydryl-labeled nucleotides, incorporation of allyl- or azide-containing nucleotides, and many other methods described in Bioconjugate Techniques (2nd Ed), Greg T. Hermanson, Elsevier (2008), which is specifically incorporated herein by reference. In some embodiments the specific binding agent has been immobilized for example on a solid support, thereby isolating the target nucleic molecule of interest. By “solid support or carrier” is intended any support capable of binding a targeting nucleic acid. Well-known supports or carriers include glass, polystyrene, polypropylene, polyethylene, dextran, nylon, amylases, natural and modified celluloses, polyacrylamides, agarose, gabbros, and magnetite. The nature of the carrier can be either soluble to some extent or insoluble for the purposes of the present disclosure. The support material may have virtually any possible structural configuration so long as the coupled molecule is capable of binding to targeting probe. Thus, the support configuration may be spherical, as in a bead, or cylindrical, as in the inside surface of a test tube, or the external surface of a rod. Alternatively, the surface may be flat such as a sheet or test strip. After capture, these end joined nucleic acid fragments are available for further analysis, for example to determine the sequences that contributed to the information encoded by the ligation junction, which can be used to determine which DNA sequences are close in spatial proximity in the cell, for example to map the three dimensional structure of DNA in a cell such as genomic and/or chromatin bound DNA. In some embodiments, the sequence is determined by PCR, hybridization of a probe and/or sequencing, for example by sequencing using high-throughput paired end sequencing. In some embodiments determining the sequence at the one or more junctions of the one or more end joined nucleic acid fragments comprises nucleic acid sequencing, such as short-read sequencing technologies or long-read sequencing technologies. In some embodiments, nucleic acid sequencing is used to determine two or more junctions within an end-joined concatemer simultaneously.

As used herein the term “specific binding agent” refers to an agent that binds substantially or preferentially only to a defined target such as a protein, enzyme, polysaccharide, oligonucleotide, DNA, RNA, recombinant vector or a small molecule. In an example, a “specific binding agent that specifically binds to the label” is capable of binding to a label that is covalently linked to a targeting probe.

In some embodiments, determining the sequence of a junction includes using a probe that specifically binds to the junction at the site of the two joined nucleic acid fragments. In particular embodiments, the probe specifically hybridizes to the junction both 5′ and 3′ of the site of the join and spans the site of the join. A probe that specifically binds to the junction at the site of the join can be selected based on known interactions, for example in a diagnostic setting where the presence of a particular target junction, or set of target junctions, has been correlated with a particular disease or condition. It is further contemplated that once a target junction is known, a probe for that target junction can be synthesized.

In some embodiments, the end joined nucleic acids are selectively amplified. In some examples, to selectively amplify the end joined nucleic acids, a 3′ DNA adaptor and a 5′ RNA, or conversely a 5′ DNA adaptor and a 3′ RNA adaptor can be ligated to the ends of the molecules can be used to mark the end joined nucleic acids. Using primers specific for these adaptors only end joined nucleic acids will be amplified during an amplification procedure such as PCR. In some embodiments, the target end joined nucleic acid is amplified using primers that specifically hybridize to the adaptor nucleic acid sequences present at the 3′ and 5′ ends of the end joined nucleic acids. In some embodiments, the non-ligated ends of the nucleic acids are end repaired. In some embodiments attaching sequencing adapters to the ends of the end ligated nucleic acid fragments.

As used herein the term “primers” refers to short nucleic acid molecules, such as a DNA oligonucleotide, which can be annealed to a complementary target nucleic acid molecule by nucleic acid hybridization to form a hybrid between the primer and the target nucleic acid strand. A primer can be extended along the target nucleic acid molecule by a polymerase enzyme. Therefore, primers can be used to amplify a target nucleic acid molecule, wherein the sequence of the primer is specific for the target nucleic acid molecule, for example so that the primer will hybridize to the target nucleic acid molecule under very high stringency hybridization conditions.

The specificity of a primer increases with its length. Thus, for example, a primer that includes 30 consecutive nucleotides will anneal to a target sequence with a higher specificity than a corresponding primer of only 15 nucleotides. Thus, to obtain greater specificity, probes and primers can be selected that include at least 5, 10, 15, 20, 25, 30, 35, 40, 45, 50 or more consecutive nucleotides.

In particular examples, a primer is at least 15 nucleotides in length, such as at least 5 contiguous nucleotides complementary to a target nucleic acid molecule. Particular lengths of primers that can be used to practice the methods of the present disclosure include primers having at least 5, at least 10, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 45, at least 50, or more contiguous nucleotides complementary to the target nucleic acid molecule to be amplified, such as a primer of 5-60 nucleotides, 15-50 nucleotides, 15-30 nucleotides or greater.

Primer pairs can be used for amplification of a nucleic acid sequence, for example, by PCR, or other nucleic-acid amplification methods known in the art. An “upstream” or “forward” primer is a primer 5′ to a reference point on a nucleic acid sequence. A “downstream” or “reverse” primer is a primer 3′ to a reference point on a nucleic acid sequence. In general, at least one forward and one reverse primer are included in an amplification reaction. PCR primer pairs can be derived from a known sequence, for example, by using computer programs intended for that purpose such as Primer (Version 0.5, © 1991, Whitehead Institute for Biomedical Research, Cambridge, MA).

Methods for preparing and using primers are described in, for example, Sambrook et al. (1989) Molecular Cloning: A Laboratory Manual, Cold Spring Harbor, New York; Ausubel et al. (1987) Current Protocols in Molecular Biology, Greene Publ. Assoc. & Wiley-Intersciences.

### Shearing Chromatin

In some aspects, the chromatin isolated from the primary cell sample is sheared for to obtain chromatin fragments for immunoprecipitation (i.e., chromatin IP (ChIP)). For example, chromatin in nuclei is sheared or chromatin that is digested, end labeled with nucleotides including biotin, and ligated is sheared. The chromatin is sheared using focused ultrasonication technology (e.g., Adaptive Focused Acoustics™ (AFA) (Covaris, Inc.)). In certain embodiments, shearing the chromatin using focused ultrasonication occurs at least twice. Other embodiments may include chromatin sheared using focused ultrasonication technology for a total of at least 30 minutes, or for a total of at least four hours.

### Immunoprecipitation of Chromatin Fragments

In example embodiments, epigenetic maps showing DNA protein-binding can be generated by immunoprecipitation of sheared chromatin fragments with antibodies specific for chromatin proteins or chromatin modifications, such as modified histones, transcription factors, chromatin modification proteins, chromatin proteins that recognize a chromatin modification, proteins involved in higher order 3D chromatin structure (e.g., loop proteins, such as cohesion and CTCF).

Chromatin Immunoprecipitation (ChIP) is used to immunoprecipitate chromatin to determine sequences bound by proteins or modified histones. ChIP-seq combines chromatin immunoprecipitation (ChIP) with massively parallel DNA sequencing to identify the binding sites of DNA-associated proteins (see, e.g., Nakato R, Sakata T. Methods for ChIP-seq analysis: A practical workflow and advanced applications. Methods. 2021; 187:44-53). Example epigenetic modifications include histone methylation, histone acetylation, DNA methylation, histone phosphorylation (e.g., serine and threonine, or tyrosine), histone ubiquitylation, histone sumoylation, histone ADP ribosylation, histone proline isomerization, histone biotinylation, and histone citrullination (see, e.g., Epigenetics, Second Edition, 2015, Edited by C. David Allis; Marie-Laure Caparros; Thomas Jenuwein; Danny Reinberg; Associate Editor Monika Lachlan; Dawson M A, Kouzarides T. Cancer epigenetics: from mechanism to therapy. Cell. 2012; 150 (1): 12-27; Syding L A, Nickl P, Kasparek P, Sedlacek R. CRISPR/Cas9 Epigenome Editing Potential for Rare Imprinting Diseases: A Review. Cells. 2020; 9 (4): 993; and Zhang Y. Transcriptional regulation by histone ubiquitination and deubiquitination. Genes Dev. 2003; 17 (22): 2733-2740).

In exemplary embodiments, chromatin is sheared and immunoprecipitated in a single reaction volume (i.e., one pot). For example, chromatin is sheared using acoustic force in a reaction volume followed by the addition of antibodies to the same reaction volume for immunoprecipitation. Immunoprecipitation can be performed by any method known in the art for chromatin immunoprecipitation (ChIP) or HIChIP (see, e.g., Collas P. The current state of chromatin immunoprecipitation. Mol Biotechnol. 2010; 45 (1): 87-100; Rosenfeld J M, Cooke T, Li Z, et al. Systematic optimization of parameters involved in preparation of chromatin and chromatin immunoprecipitation (ChIP) workflow. Epigenetics Chromatin. 2013; 6 (Suppl 1): P122; and Mumbach M R, Rubin A J, Flynn R A, et al. HiChIP: efficient and sensitive analysis of protein-directed genome architecture. Nat Methods. 2016; 13 (11): 919-922).

### Sequencing

In a non-limiting example, the immunoprecipitated chromatin fragments are purified and DNA analysis is performed. In example embodiments, DNA analysis is selected from quantitative PCR or targeted sequencing and next-generation sequencing.

As used herein the term “DNA sequencing” refers to the process of determining the nucleotide order of a given DNA molecule. In certain embodiments, the sequencing can be performed using automated Sanger sequencing. In certain embodiments, sequencing comprises high-throughput (formerly “next-generation”) technologies to generate sequencing reads from the one or more nucleic acid fragments. In DNA sequencing, a read is an inferred sequence of base pairs (or base pair probabilities) corresponding to all or part of a single DNA fragment. A typical sequencing experiment involves fragmentation of the genome into millions of molecules or generating complementary DNA (cDNA) fragments, which are size-selected and ligated to adapters. The set of fragments is referred to as a sequencing library, which is sequenced to produce a set of reads. Methods for constructing sequencing libraries are known in the art (see, e.g., Head et al., Library construction for next-generation sequencing: Overviews and challenges. Biotechniques. 2014; 56 (2): 61-77; Trombetta, J. J., Gennert, D., Lu, D., Satija, R., Shalek, A. K. & Regev, A. Preparation of Single-Cell RNA-Seq Libraries for Next Generation Sequencing. Curr Protoc Mol Biol. 107, 4 22 21-24 22 17, doi: 10.1002/0471142727.mb0422s107 (2014). PMCID: 4338574).

A “library” or “fragment library” may be a collection of nucleic acid molecules derived from one or more nucleic acid samples, in which fragments of nucleic acid have been modified, generally by incorporating terminal adapter sequences comprising one or more primer binding sites and identifiable sequence tags. In certain embodiments, the library members (e.g., genomic DNA fragments, cDNA) may include sequencing adaptors that are compatible with use in, e.g., Illumina's reversible terminator method, long read nanopore sequencing, Roche's pyrosequencing method (454), Life Technologies' sequencing by ligation (the SOLID platform) or Life Technologies' Ion Torrent platform. Examples of such methods are described in the following references: Margulies et al (Nature 2005 437:376-80); Schneider and Dekker (Nat Biotechnol. 2012-4-10; 30 (4): 326-8); Ronaghi et al (Analytical Biochemistry 1996 242:84-9); Shendure et al (Science 2005 309:1728-32); Imelfort et al (Brief Bioinform. 2009 10:609-18); Fox et al (Methods Mol. Biol. 2009; 553:79-108); Appleby et al (Methods Mol. Biol. 2009; 513:19-39); and Morozova et al (Genomics. 2008 92:255-64), which are incorporated by reference for the general descriptions of the methods and the particular steps of the methods, including all starting products, reagents, and final products for each of the steps.

Roadblocks for High Resolution/High-throughput 3D Chromatin Analysis include (1) sample input requirements, (2) scalability for automation, (3) processing time, and (4) the cost per sample analysis. Mapping the 3D epigenetic landscape requires high cell numbers and sample preparation is hard to standardize across different cell types. Previous chromatin immunoprecipitation (ChIP) and high throughput ChIP (HiChIP) workflows have been difficult to scale down to sample volumes suitable for 96-well formats and one-pot automation pipelines. Previous ChIP and HiChIP workflows are lengthy protocols of 3-4D with a lot of hands-on time, limiting sample throughput required for epigenetic screening. And lastly, high costs and sequencing depth limit access to end users outside of major academic centers and the industry.

Disclosed herein are methods of epigenomic mapping, even for low cell input samples, using acoustic enhanced immunoprecipitation. As shown in the Examples, high resolution mapping of the epigenetic landscape was possible with input of as low as 20,000 cells. Also disclosed herein are methods of isolating chromatin from small volume and small number of cells using acoustic enhanced immunoprecipitation.

Specifically, Covaris®'s Adaptive Focused Acoustics® (AFA®) focused ultrasonication technology was implemented to miniaturize sample volumes for both chromatin shearing and immunoprecipitation in a 96-well format. This approach enables a standardized, robust, automatable, half-day workflow for both ChIP and Hi-ChIP applications. In some aspects, the ChIP workflows can be completed in a single day, while the HiChIP can be completed in just 1.5 days.

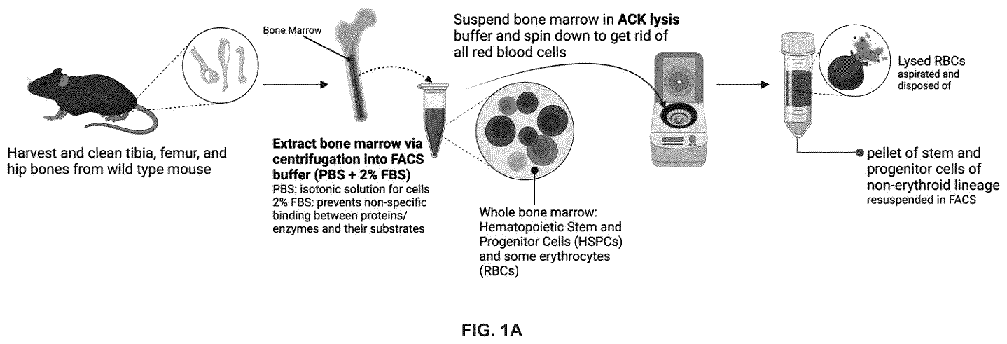

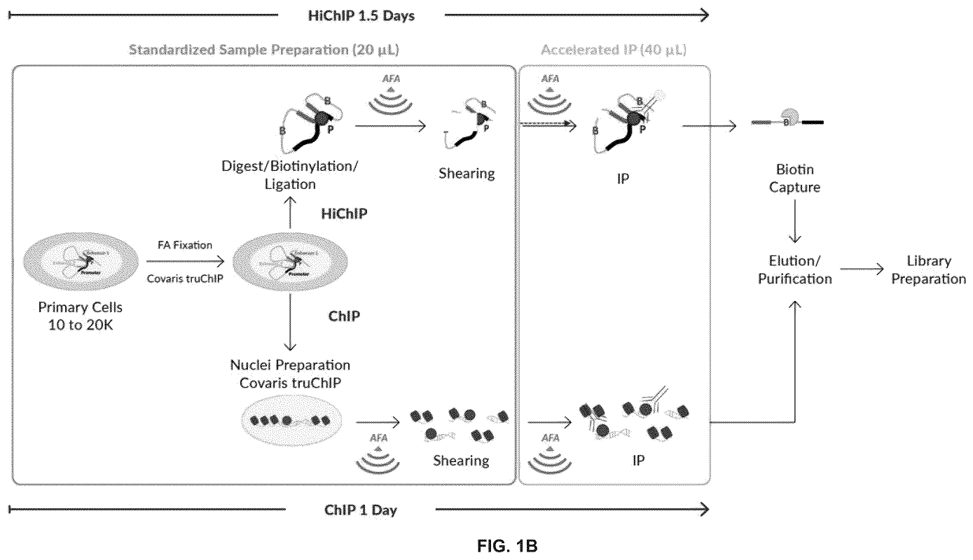

The method comprises providing a primary cell sample comprising at least 5,000 primary cells; isolating chromatin from the primary cell sample; shearing the isolated chromatin with AFA, preferably twice; and the immunoprecipitating the sheared chromatin with chromatin binding agent. The chromatin binding agent may be a histone or a DNA binding protein. In some aspects, the sheared chromatin then undergoes library preparation for sequencing. FIG. 1B depicts an exemplary schematic for high input and regular ChIP processing of hematopoietic cells. The ChIP and HiChIP workflows shown in FIG. 1B demonstration optimization for 96-well formats and one-pot automation pipelines.

In some implementations, the primary cell sample comprises 5,000 to 500,000 cells, 5,000 to 50,000 primary cells, 5,000 to 20,000 primary cells, and/or 10,000 to 20,000 primary cells. For example, the primary cell sample comprises 5,000 to 50,000 bone marrow cells. In some aspects, the volume of the isolated chromatin from the primary cell sample used for shearing with AFA is less than 1 ml, for example, less than 50 μl, less than 40 μl, less than 30 μl, or about 20 μl. For immunoprecipitating the sheared chromatin, the volume of the sheared chromatin is less than 1 ml, for example, less than 50 μl, less than 40 μl, less than 30 μl, or about 20 μl. Miniaturizing reactions can reduce sample prep costs >5× and sequencing cost. The described method reduces reaction volumes and costs without reducing yield of sheared chromatin and immunoprecipitated chromatin.

As shown in the Example, AFA ChIPseq was applied to flow sorted hematopoietic stem and progenitor cells (HSPC) from individual mice. A range of epitopes including major histone modifications (H3K9me1/2/3, H3K27ac) and factors (CTCF, cohesin) was studied. Interestingly, the time required of AFA pulse for identifying epitope-dependent differences differed depending on the histone modification or factors studied.

Also shown in the Examples, overlap of peaks called by MACS2 was consistent among the various cell inputs as well as when compared with gold standard CTCF ChIPseq-data previously performed on pooled samples from previously published data. The low cell input capability permitted deconvolution of dynamic epigenetic changes in protein binding and chromatin structure during normal lineage specification of hematopoietic stem cells. Epigenetic remodeling of these sites was consistent with established gene programs essential for myeloid differentiation. Data were found to be in alignment with standard ChIPseq- and Hi-C datasets generated from similar cell populations.

The simplified and significantly shortened (Hi) ChIP-seq processes showcased in this study is highly beneficial for both research and clinical diagnostic applications owing to their requirements for using 96-well plate format and their ability to become integrated into automated workflows. Taken together, this technology and workflow represents a high throughput epigenetic system for small cell numbers of cells with a multitude of applications enabling laboratories focused on cell biology and translational research.

### Examples

The disclosure is further illustrated by the following examples that should not be construed as limiting. The contents of all references, patents, and published patent applications cited throughout this application, as well as the Figures, are incorporated herein by reference in their entirety for all purposes.

### Example 1. Mouse Hematopoietic Stem and Progenitor Cells

The claimed method was applied to flow sorted hematopoietic stem and progenitor cells (HSPC) from individual mice. A range of epitopes including major histone modifications (H3K9me1/2/3 or H3K27ac) and factors (CTCF or cohesin) was studied using chromatin processed according to the claimed method. Interestingly, epitope-dependent differences were found in in AFA-enhanced binding with CTCF saturating at 30 minutes, whereas H3K27ac required 4 hours of AFA pulse to facilitate chromatin binding.

Additionally, low input in vitro and in vivo samples were evaluated. Limited dilution of lineage negative HSPCs ranging from 500,000 to 5,000 cells had comparable sheared chromatin yield and immunoprecipitated material. Overlap of peaks called by MACS2 was consistent among the various cell inputs as well as when compared with gold standard CTCF ChIPseq-data previously performed on pooled samples from previously published work. The low cell input capability permitted deconvolution of dynamic epigenetic changes in protein binding and chromatin structure during normal lineage specification of hematopoietic stem cells. Epigenetic remodeling of these sites was consistent with established gene programs essential for myeloid differentiation. Data were found to be in alignment with standard ChIPseq- and Hi-C datasets generated from similar cell populations.

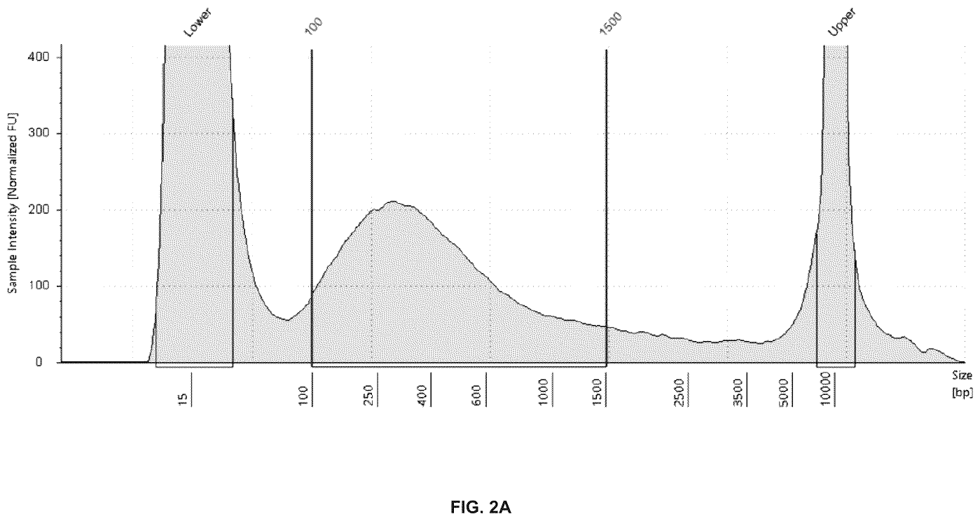

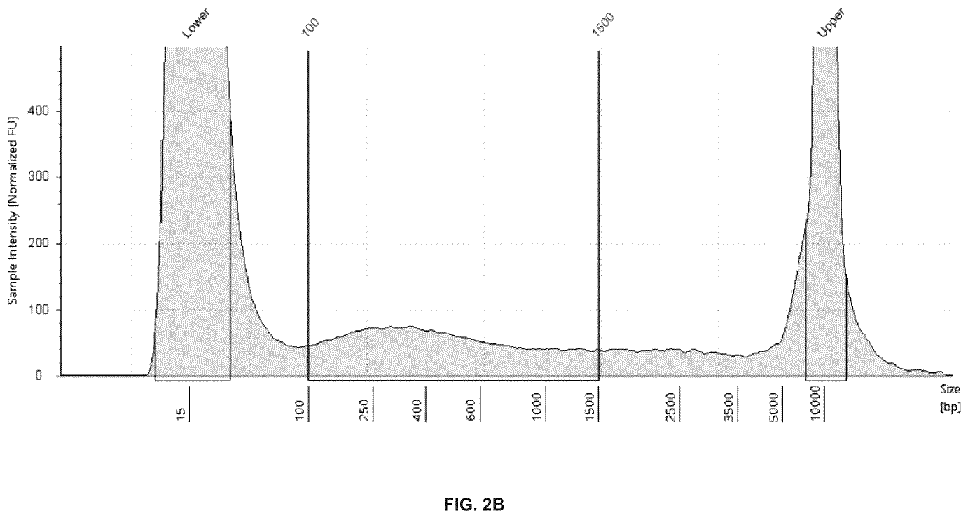

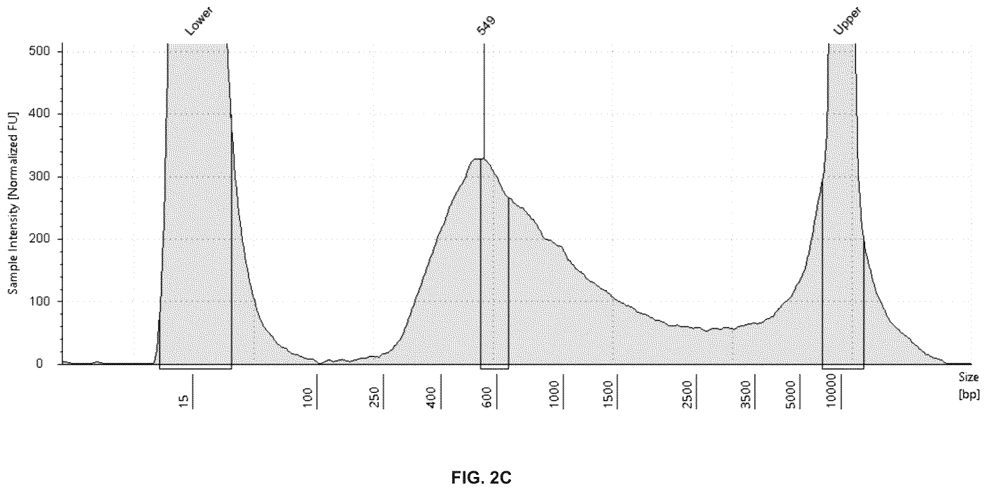

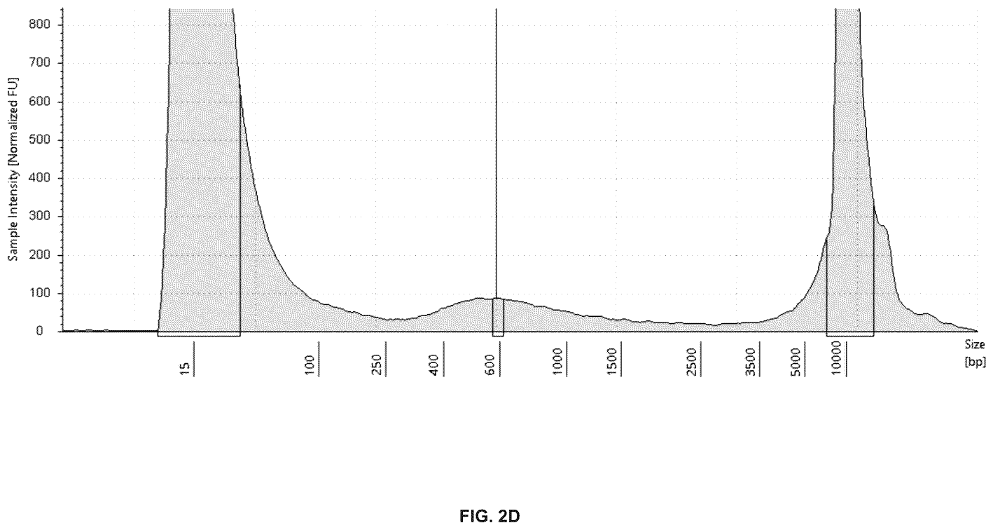

As shown in FIGS. 2A-2D, truChIP® chemistry in combination with mechanical chromatin shearing on the 96-well Covaris® LE220Rsc Focused-ultrasonicator enables reproducible chromatin shearing from limited primary cell material. Different primary cell populations were subjected to the same chromatin shearing settings yielding ideal shearing results enabling a high sample input flexibility in the same 96-well experimental run.

Hematopoietic stem cells have specific enhancer promoter interaction bounded by CTCF that is not present after lineage specific commitment. As Simultaneous CTCF and LOOP structure is captured by CTCF HiChIP, which confirms the single assay (Hi-C findings).

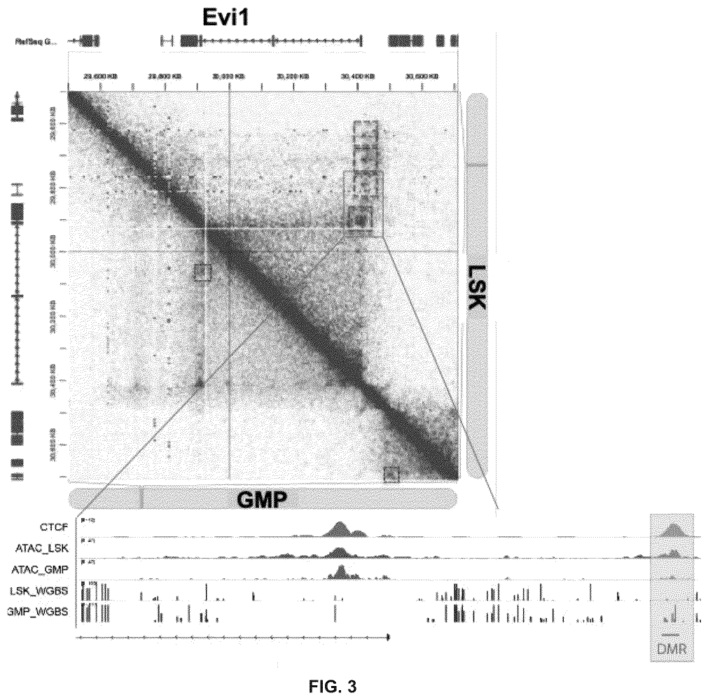

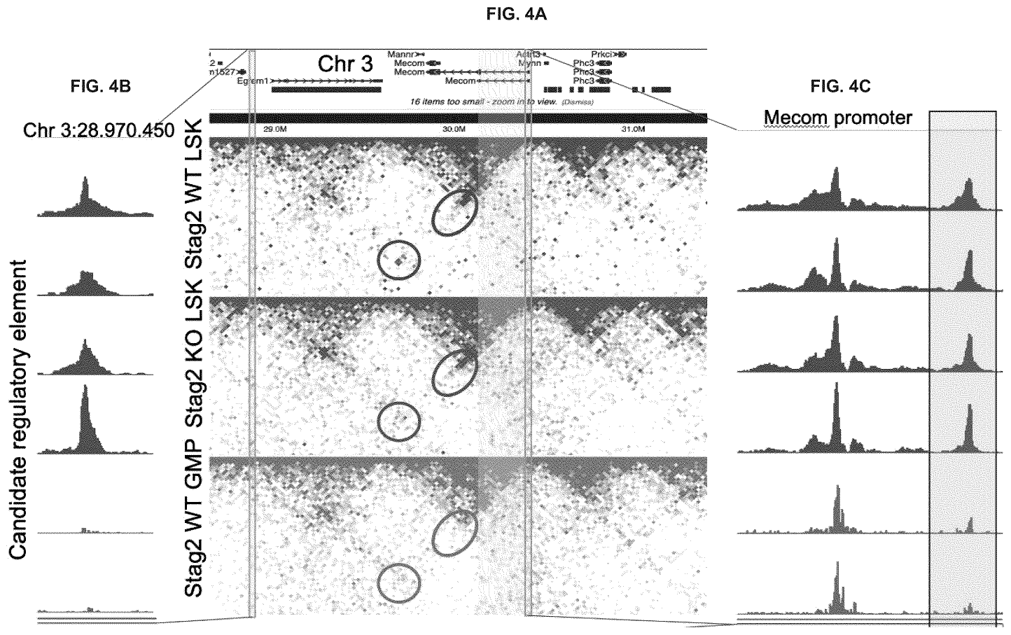

Dynamic changes are evident at the Mecom/Evil enhancer, which loses ATAC accessibility and gains a differentially methylated region at a CTCF site within the Mecom/Evil enhancer (FIG. 3). Low input CTCF HiChIP validates CTCF ChIP and Hi-C. The Mecom/Evil locus identified in FIGS. 4A and 4B was able to be captured by a single assay with 10,000 to 20,000 primary cell input, which is a major technological innovation.

### Example 2. Mouse Bone Marrow Cells

The AFA technology can be successfully applied with a reduced sample volume for both shearing (from 1 ml to 20 μl) and immunoprecipitation (from 1 ml to 40 μl). Using this optimized protocol with 5,000 to 50,000 mouse bone marrow cells in the primary cell sample, it was possible to increase yield in both sheared (21.8 μg) and immunoprecipitated (8 μg) chromatin and observe improved signal to noise ratio.

To profile chromatin interactions changes during stem to myeloid transition, CTCF Hi-ChIP and CTCF ChIPseq was performed on sorted LSK (Lineage-Sca1+Kit+) cells and granulocyte-macrophage progenitors (Lineage-Sca1-Kit+Cd34+Fcy+). Compared to conventional Hi-C, CTCF Hi-ChIP recapitulated topological looping with higher read count, less input material, and lower sequencing depth (90×106 Hi-ChIP reads vs. 800×106 Hi-C reads). Overlaying ATAC-Seq, CTCF ChIP-Seq, and bisulfite sequencing, no promoter-based changes in accessibility or methylation of Mecom, an HSC dependent gene, was observed. Yet, dynamic changes at 5′ UTR and 1.7 MB regulatory elements were observed during myeloid commitment consisted with locus control of Mecom repression.

More specifically, this disclosure, its aspects and embodiments, are not limited to the specific material types, components, methods, or other examples disclosed herein. Many additional material types, components, methods, and procedures known in the art are contemplated for use with particular implementations from this disclosure. Accordingly, for example, although particular implementations are disclosed, such implementations and implementing components may comprise any components, models, types, materials, versions, quantities, and/or the like as is known in the art for such systems and implementing components, consistent with the intended operation.

Many additional implementations are possible. Further implementations are within the CLAIMS.

We claim:

1. A method of assessing epigenetic regulation in a tissue comprising:
isolating primary cells from the tissue to produce a primary cell sample, wherein the primary cell sample comprises at least 5,000 primary cells;
isolating chromatin from the primary cell sample;
shearing the isolated chromatin using focused ultrasonication;
immunoprecipitating the sheared chromatin; and
isolating the sheared chromatin for DNA analysis.

2. The method of claim 1, wherein the primary cell sample comprises 5,000 to 500,000 primary cells.

3. The method of claim 1, wherein the primary cell sample comprises 10,000-20,000 primary cells.

4. The method of claim 1, wherein the shearing using focused ultrasonication includes application of the focused ultrasonication at least twice.

5. The method of claim 1, wherein the primary cells are hematopoietic cells.

6. The method of claim 1, wherein the primary cell sample is isolated from bone marrow.

7. The method of claim 6, wherein the tissue is from a mammalian subject.

8. A method of isolating chromatin comprising:
isolating primary cells from tissue to produce a primary cell sample, wherein the primary cell sample comprises at least 5,000 primary cells;
isolating chromatin from the primary cell sample;
shearing the isolated chromatin using focused ultrasonication technology at least twice;
immunoprecipitating the sheared chromatin; and
isolating the sheared chromatin for DNA analysis.

9. The method of claim 8, wherein the primary cell sample comprises 10,000-20,000 primary cells.

10. The method of claim 8, wherein the primary cell sample comprises at least 20,000 primary cells.

11. The method of claim 8, wherein the primary cell sample comprises 5,000-50,000 primary cells.

12. The method of claim 8, wherein the primary cells are hematopoietic cells.

13. The method of claim 8, wherein the primary cell sample is isolated from bone marrow of a mammalian subject.

14. A method isolating chromatin comprising:
isolating primary cells from bone marrow to produce a primary cell sample, wherein the primary cell sample comprises at least 5,000 bone marrow cells;
isolating chromatin from the primary cell sample;
shearing the isolated chromatin using focused ultrasonication technology at least twice;
immunoprecipitating the sheared chromatin; and
isolating the sheared chromatin for DNA analysis.

15. The method of claim 14, wherein the primary cell sample comprises 10,000-20,000 primary cells.

16. The method of claim 14, wherein the primary cell sample comprises at least 20,000 primary cells.

17. The method of claim 14, wherein the primary cell sample comprises 5,000-50,000 primary cells.

18. The method of claim 14, wherein the DNA analysis is selected from quantitative PCR or next-generation sequencing.

19. The method of claim 14, wherein the isolated chromatin is sheared using focused ultrasonication technology for a total of at least 30 minutes.

20. The method of claim 14, wherein the isolated chromatin is sheared using focused ultrasonication technology for a total of at least four hours.
